(19) United States
(12) Patent Application Publication
(10) Pub. No.: US 2016/0310791 A1
(43) Pub. Date: Oct. 27, 2016

(54) Activity and Exercise Monitoring System

(71) Applicant: Gen-Nine, Inc., Patchogue, NY (US)

(72) Inventor: Mark A. FAUCI, Louisville, KY (US)

(21) Appl. No.: 15/136,320

(22) Filed: Apr. 22, 2016

Related U.S. Application Data
(60) Provisional application No. 62/151,652, filed on Apr. 23, 2015.

(51) Int. Cl. A63B 24/00; A61B 5/01; A61B 5/00; A61B 5/024

(52) U.S. Cl. CPC A63B 24/0075; A63B 24/0062; A61B 5/02433; A61B 5/015; A61B 5/486; A61B 5/6801

(57) ABSTRACT

The present invention provides systems and methods for providing physical therapy exercise regimens and detecting electromagnetic radiation associated with movement and physiology.

### CROSS REFERENCE

This Application claims the benefit of U.S. Provisional Application No. 62/151,652, filed Apr. 23, 2015, which is incorporated herein by reference in its entirety.

### BACKGROUND OF THE INVENTION

An at-home physical therapy program comprising about 10 to about 15 minutes of balance, exercise, and strength training can slow the functional decline of individuals, especially the elderly and physically frail. A regular regimen of structured exercise or physical therapy can improve measures of mobility and fitness, for example, strength and aerobic capacity. The positive effects of structured exercise can occur in both chronically-ill and healthy adults. Exercise can also produce improvements in gait and balance, and other long-term functional benefits, and decrease pain symptoms, for example, in arthritis.

Exercise promotes bone mineral density, and thereby, decreases fracture risk. Exercise can also counteract key risk factors for falls, such as poor balance, and consequently, reduce the risk of falling. Falls can cause traumatic brain injury, and fall-related head injuries can make individuals, especially those taking anticoagulants, susceptible to intracranial hemorrhage. However, practical and cost-related limitations can constrain the dissemination of this type of regimen in the home-care environment.

### INCORPORATION BY REFERENCE

Each patent, publication, and non-patent literature cited in the application is hereby incorporated by reference in its entirety as if each was incorporated by reference individually.

### SUMMARY OF THE INVENTION

In some embodiments, the invention provides a method comprising: a) receiving by a computer system data associated with a first electromagnetic signal from a subject's body, wherein the data associated with the first electromagnetic signal is associated with a gesture of the subject; b) receiving by the computer system data associated with a second electromagnetic signal from the subject's body, wherein the data associated with the second electromagnetic signal is associated with a physiological characteristic of the subject; c) determining by a processor of the computer system based on the data associated with the first electromagnetic signal from the subject's body and the data associated with the second electromagnetic signal from the subject's body a suitable exercise regimen for the subject; and d) outputting the suitable exercise regimen on an output device.

### BRIEF DESCRIPTION OF THE DRAWINGS

FIG. 1 illustrates the Activity and Exercise Monitoring System (AEMS) clinical interface showing the GRS image (left) and the DIRI image (right).

FIG. 2 illustrates the AEMS user interface providing audio/visual feedback corresponding with the user exercise regimens.

FIG. 3 illustrates the AEMS Home User Module providing multispectral imaging, NIR/GRS, and LWIR/DIRI sensors.

FIG. 4 illustrates the AEMS cloud server connecting the Home User Module to the AEMS clinical systems, and other systems through application program interfaces (APIs).

FIG. 5 shows the sequence of steps in which the AEMS can be used in combination with a monitoring device.

FIG. 6 shows the sequence of steps in which the object detector module of the AEMS identifies objects that a user wants to track.

FIG. 7 shows a diagram for training a gesture-recognition system (GRS).

FIG. 8 illustrates emission detection using long-wave infrared imaging (LWIR).

FIG. 9 shows the relationship between distance and photon count using a LWIR detector.

### DETAILED DESCRIPTION OF THE INVENTION

Presented herein are systems and methods comprising sensors that detect electromagnetic radiation associated with human movement and physiology. When combined with network technologies and structured individualized exercise programs of various formats, the invention can provide an on-demand exercise regimen. The invention can be used in the home or in other environments.

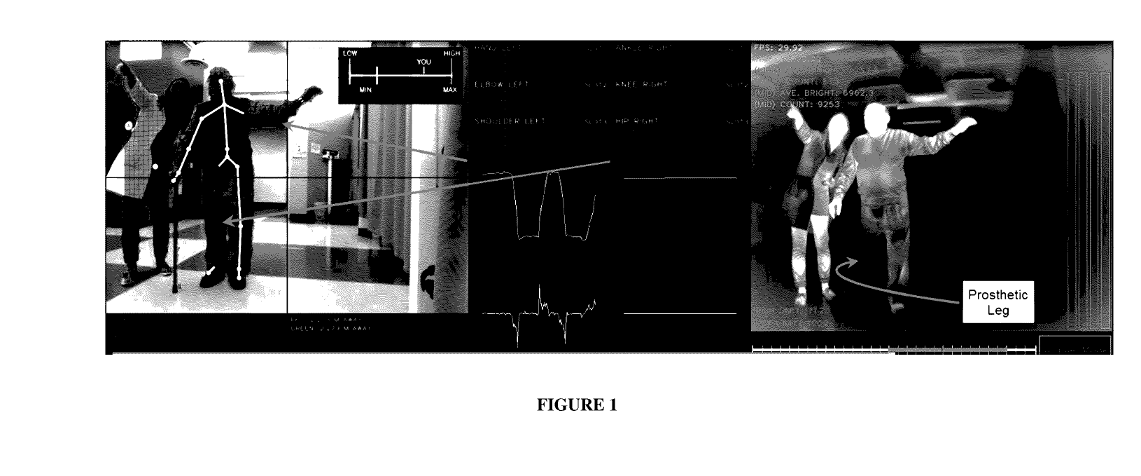

In some embodiments, the invention comprises gesture-recognition system (GRS) and dynamic infrared imaging (DIRI) combined into a single module (FIG. 1); a network system for delivery of information; and a system of structured exercise programs. These exercise programs can be delivered remotely to the home or other environments. Movement can be monitored in real-time or recorded for analysis by researchers.

The systems herein can combine near-infrared/gesture-recognition (NIR/GRS) technology with long-wave infrared/dynamic infrared imaging (LWIR/DIRI) technology into a single multi-spectral module that is more effective than either sensor technology alone for monitoring movement and physiology.

The Activity and Exercise Monitoring System (AEMS) clinical interface can display a GRS image (left) and a DIRI image (right) as illustrated in FIG. 1. The sensitivity of DIRI (right) is highlighted by revealing a prosthetic leg that is not visible using NIR (left). Fusing these data streams provides concurrent information about both activity and corresponding physiological changes measured as changes in skin temperature or heart rate, which can be measured at a distance by analyzing changes in infrared emissions. In some embodiments, physiological changes can include, for example, changes in temperature, heart rate, breathing rate, blood flow, perspiration, exercise intensity, muscle contraction, muscle relaxation, muscular strength, endurance, cardiorespiratory fitness, body composition, and flexibility.

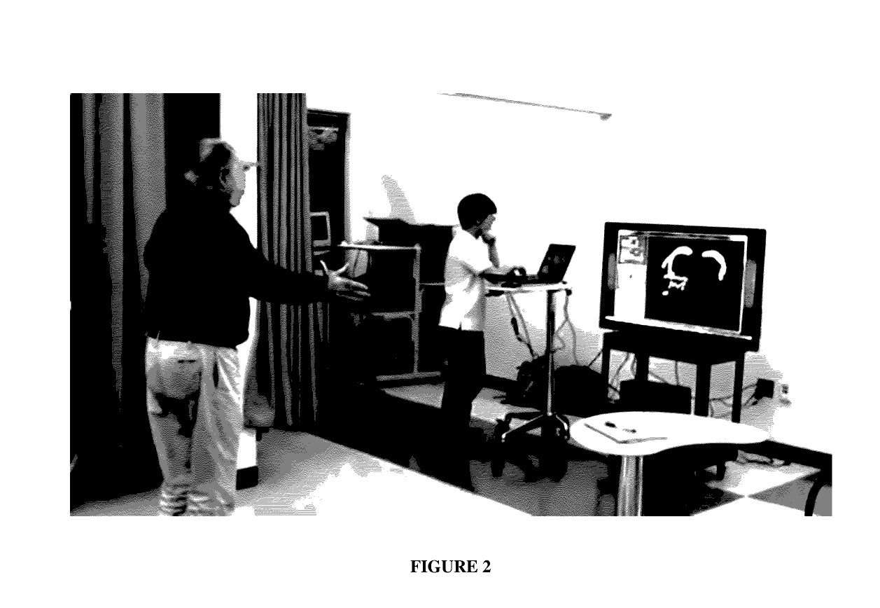

As illustrated in FIG. 2, gamification methods can be used to make user interaction with this system more enjoyable and motivational. A wearable tracking device, including, for example, a human activity monitoring (HAM) system, can be used for monitoring of the user; detecting the need for exercise, including, for example, through a fall risk assessment; making a recommendation for an exercise regimen; and further monitoring of the user. The process can be repeated in whole or in part based on the needs and interests of the user. In some embodiments, the invention can comprise a method of identifying targets and measuring X-Y-Z position and movement using electromagnetic radiation imaging, including, for example, passive LWIR/DIRI infrared imaging. The AEMS user interface gamification features provide audio/visual feedback corresponding with the user exercise regimens to provide an engaging experience. The user can partake in a number of activities including “painting” and “music conducting” by simply moving their bodies alone, with others in the room, or through virtual presence.

In some embodiments, the invention comprises the tracking of human movement and physiological changes as part of a physical therapy or structured exercise system. The physical therapy or structured exercise can be monitored by a remote clinical observer, for example, a physical therapist. The invention can be used on a wide variety of age groups in the home or other environments, including, for example, elderly individuals in a home-care environment.

In addition to providing health benefits to users by facilitating at-home exercise programs, the system presented herein can also provide researchers and clinicians with an exercise physiology research platform. The integrated network can have other benefits including, for example, promoting social contact and interaction among the elderly by providing a platform that permits users located at different locations to join in a single virtual group exercise program, as well as promoting other social interactions through a similar hardware/software infrastructure.

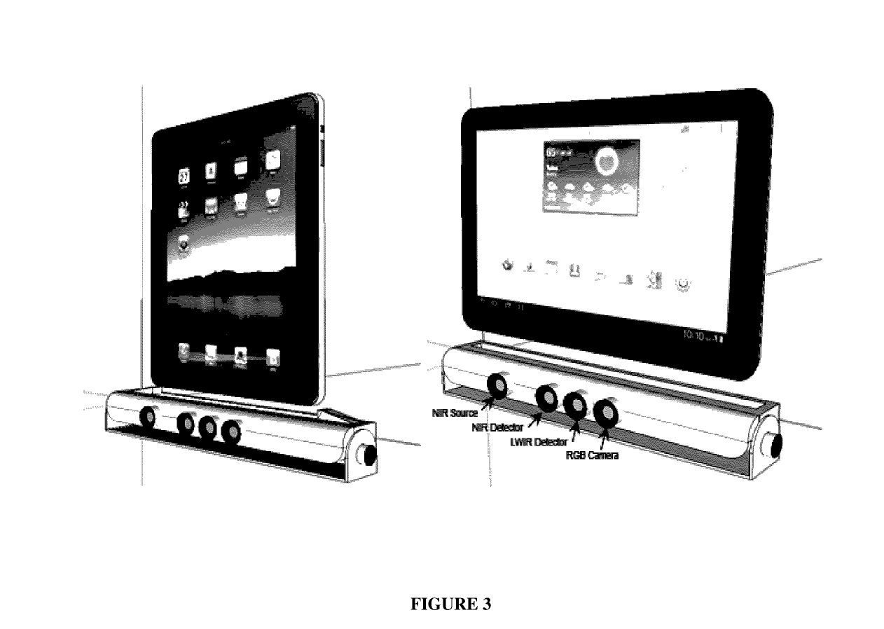

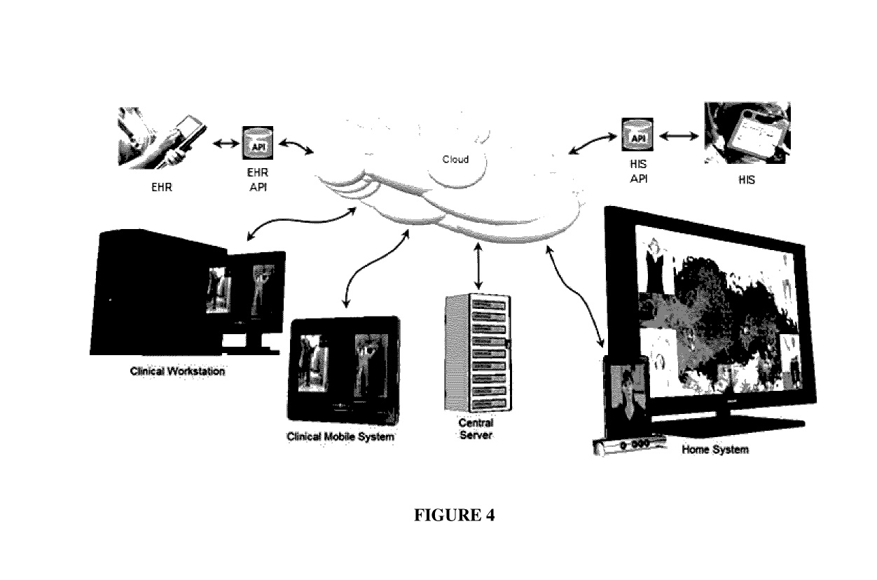

In some embodiments, the invention comprises the following components: a multi-spectral portable module that comprises an NIR/GRS imaging sensor with a NIR light source; a LWIR/DIRI imaging sensor; a visible spectrum imaging sensor; a microphone; a speaker; a wired or wireless display interface, for example, a high-definition television or smart mobile device; an algorithm that analyzes body movement and physiological response in real-time; a network application running on a remote server that can provide the exercise instruction management functions, data collection, storage, analysis, virtual presence, and data distribution functions; and an application program interface (API) for individuals to track and analyze user activity and physical health in real-time or retrospectively (FIGS. 3 and 4). The AEMS cloud server connects the Home User Module to the AEMS clinical systems and other systems through APIs (FIG. 4).

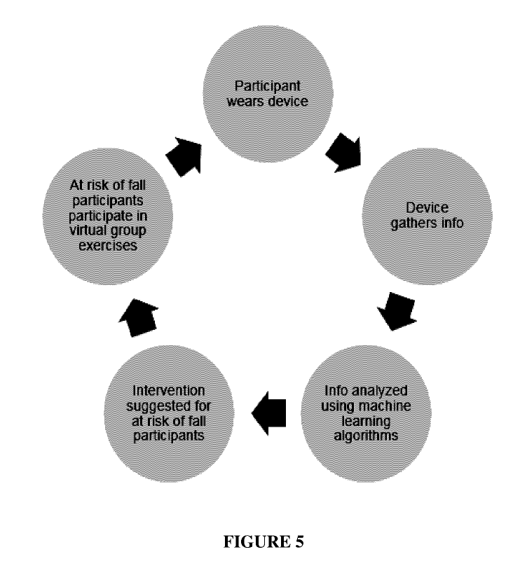

In some embodiments, the invention can be used in conjunction with other devices. In a non-limiting example, an elderly user wears the HAM device. The device can gather and analyze information recorded by the system as shown in FIG. 5. First, the HAM device can gather activity information about the user including, for example, number of steps taken, distance walked or ran, heart rate, caloric intake, and sleep patterns. The activity information can then be analyzed using machine learning algorithms, which can assess the overall activity of the user to predict whether there is a significant risk for a fall. The device can then suggest an intervention for the at-risk-of-fall users. Using the invention, the user can then engage in an exercise regimen using a system of the disclosure designed to reduce the risk of falling. The at-risk-of-fall users can also participate in virtual group exercises with other users of the invention. The cycle of monitoring, analysis, and exercise can continue in an iterative manner. For example, feedback from the HAM device can direct the need for an exercise regimen described by the invention. The HAM device can then analyze the results, thereby determining the post-activity risk. If the initial activity is insufficient, further recommendations can be made. The HAM device can continue to monitor the user to determine whether future risks increase. In some embodiments, the invention can track the overall improvement or decline in physical health of the user. The invention can also transmit the information recorded and presented by the HAM device to other individuals, for example, health care professionals or researchers, for further analysis. Using AEMS in combination with a monitoring device can yield very powerful synergies by providing a feedback loop of progress for the user or others.

The GRS process of tracking an object comprises two steps. First, the process can teach the system to detect the specific object(s) in the field that the system is evaluating. Given an image, the system can find out the position and scale of all objects of a given class. Second, the process can perform the functions required to calculate the position and path of the identified object(s) in X-Y-Z space.

Machine-learning is a branch of artificial intelligence and pertains to the construction and study of systems that can learn from data without being explicitly programmed to perform the specific functions for which they were designed. The core of machine-learning deals with representation and generalization. Representation of data instances and functions evaluated on these instances are part of machine-learning systems.

Applying machine-learning techniques to object tracking can allow the determination of the current location and path of one or more objects in the visual field of an image. All digital images consist of an array of pixels arranged in X-Y space. These frames consist of a certain number of pixels arranged in the X and Y directions. For example, 1024×768 means the width (X) is comprised of 1024 pixels and the height (Y) is comprised of 768 pixels. Moving video images consist of multiple numbers of these frames captured over a period of time, for example, 30 frames per second. In any single frame, objects can appear, and as the video progresses these objects can continue to occupy the same X-Y position in each frame or move in any direction as a result of being located in a different X-Y position on succeeding frames.

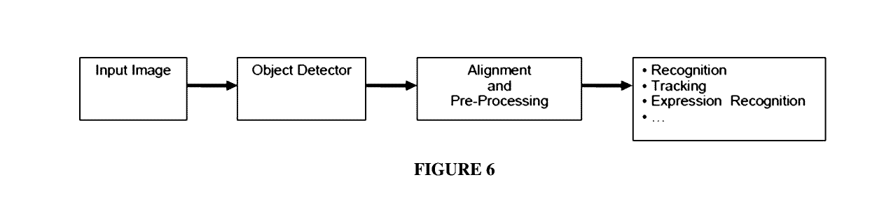

As illustrated in FIG. 6, an input image can be detected by an object detector. Then, the information received from the input image can undergo alignment and pre-processing so that the system can continuously recognize and track the object of interest.

The object detector module is the first module needed for object recognition. The process of tracking involves first teaching the system to identify the object(s) that the user wants to track and then training the system to recognize the object(s) even if the appearance, size, or shape of the object(s) can change significantly during the video sequence.

The first part of this process, teaching the system to recognize the object, involves reducing the object to its digital characteristics. This process can include analyzing object color characteristics, shape, brightness, or any combination of the above. For example, the system can use a cascade classifier method to identify the objects.

Training the cascade classifier includes preparation of training data and running a training application. Both Haar-like (Viola2001) and Local Binary Patterns (LBP—Liao2007) features can be used. A Haar-like feature considers adjacent rectangular regions at a specific location in a detection window, sums up the pixel intensities in each region, and calculates the difference between these sums. This difference is then used to categorize subsections of an image. For example, for an image database with human faces, the region of the eyes is darker than the region of the cheeks. Therefore, a Haar-like feature for face detection is a set of two adjacent rectangles above the eye and cheek regions. The position of these rectangles is defined relative to a detection window that acts as a bounding box to the target object (the face in the above example).

The LBP is a simple local descriptor which generates a binary code for a pixel neighborhood, which comprises a given pixel and those pixels adjacent to the edges in two- or three-dimensional space. A LBP can focus either on the definition of the location where gray value measurements are taken, or on post-processing steps that improve discriminability of the binary code. Unlike Haar-like features, LBP features are integer values, so both training and detection with LBP features are several times faster than with Haar-like features. A LBP-based classifier can be trained to provide similar quality as a Haar-based classifier, thereby permitting similar detection accuracy with reduced processing time. LBP and Haar-like detection quality depends on training: the quality of both the training dataset and the training parameters.

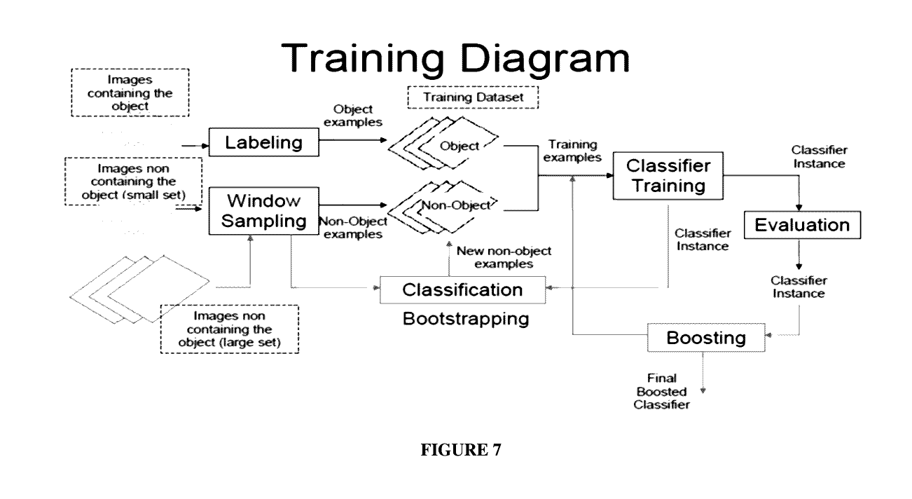

FIG. 7 illustrates the process of dataset training of a GRS system. The training requires two sets of samples: positive samples (object images; “images containing the object”) and negative samples (non-object images; “images not containing the object (small set)”). The set of positive samples can be prepared using an application utility, whereas the set of negative samples can be prepared manually. First, object images can be labeled by the labeling module to differentiate from the non-object samples (small and large set), which are instead processed by the window sampling module. Both object and non-object samples, collectively known as the training dataset, can be classified (“bootstrapped”) by the classifier training module. New non-object examples can also be classified by the classifier module. The classifier training module can differentiate the object samples from the non-object samples. Negative samples can be removed from arbitrary images that do not contain the detected objects. Then, the object samples can undergo evaluation and boosting. This process of evaluation and boosting can cycle again when new object samples are received by the classifier training module. Instead of evaluation and boosting, the non-object samples can undergo classification and bootstrapping. This process of classification and bootstrapping can also cycle again when new non-object samples are received by the classifier training module.

Negative samples can be enumerated in a special file. Data can be stored in a text file in which each line contains an image filename (relative to the directory of the description file) of the negative sample image. This file can also be created manually. Negative samples and sample images can also be called background samples or background sample images.

Positive samples can be created from a single image with object(s) or from a collection of previously annotated images. Larger numbers of images presenting a diverse set of presentation scenarios offer the best training outcome. For example, a single object image can contain a company logo. However, a larger set of positive samples can be created from the given object image by random rotating, changing the logo intensity, as well as placing the logo on arbitrary backgrounds. To achieve very high recognition rates (greater than about 90%) hours or days can be required for each iteration of training during the development process.

Once the system can identify the target, algorithms were developed to define the position of the identified object in 3D space. First, the object was placed on the X-Y axis using methods that utilize the perceived position of the object relative to the absolute position of each pixel in the pixel array that defines a single field of each frame. Identifying the Z-axis position, however, can be more complex and can utilize specialized hardware. One 3D measurement technology, called light coding, works by coding the scene with NIR light, which is not visible to the human eye. A complementary metal oxide semiconductor (CMOS) image sensor can read the coded light back from the scene.

Light coding works by projecting a pattern of IR dots from the sensor and detecting those dots using a conventional CMOS image sensor with an IR filter. The pattern can change based upon objects that reflect the light. The dots can change size and position based on how far the objects are from the source. The hardware takes the results from the image sensor and determines the differences to generate a depth map. An example resolution of the depth map can be 1024×768, but CMOS sensors can have a much higher resolution. The image resolution that can be captured by the hardware can be 1600×1200, and can provide a depth map. The chip can manage the computational load of identifying the dots and translating their state into a depth value. With the implementation in the hardware, the chip can maintain.

Investigations presented herein indicated the system can report a depth of at least about 0.8 meters to about 1.5 meters. The field of vision can be about a 58° horizontal x about a 45° vertical rectangular cone. Investigations presented herein further indicated sensitivity to numerous factors, including ambient light, reflectance and angle of surfaces in the scene, as well as the amplitude of the reflected light. As a result, these systems can be limited for use in only close-proximity applications, for example, moving a cursor on a screen that is within about a one-half meter of the detector.

In some embodiments, the invention can employ a GRS module that uses an active imaging system of an NIR light source and detector. Motion tracking is achieved by encoding the light source with information that is projected onto the scene and then reflected back to the detector, which then analyzes the reflected light to detect the X-Y-Z position and changes in position.

In some embodiments, the invention comprises a passive, DIRI module. In some embodiments, no artificial light source is used with this module. The subject, for example, a human user, is the source of infrared light. Human tissue emits electromagnetic radiation (from about 8 μm to about 10 μm in wavelength). In some embodiments, the imaging sensor detects this electromagnetic radiation to produce an image. In some embodiments, the invention can distinguish the object from the background and then measure the X-Y-Z position and changes in position. This method presented herein can be used over greater depths and angles as compared with GRS imaging alone (as described above). In some embodiments, the method can also be unaffected by ambient lighting conditions.

In some embodiments, the principal object that the system detects is a human subject, or some part of a human subject, for example, the face, hands, or fingers. In some embodiments, the system can detect movement of a limb of the subject, including, for example, the arms and legs. In some embodiments, the system can detect movement of a body part of the subject including, for example, the hands, fingers, toes, shoulders, elbows, knees, hips, waist, back, chest, torso, head, and neck.

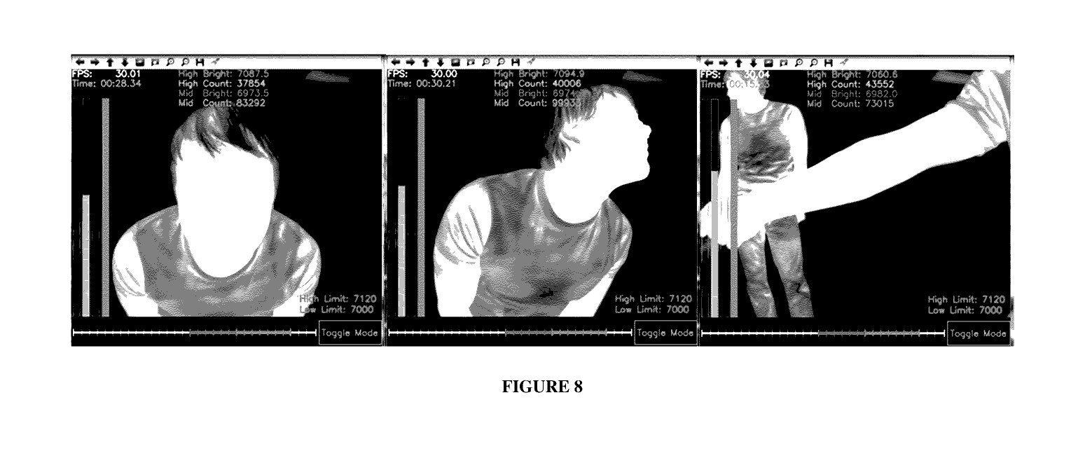

A LWIR/DIRI system was used to detect electromagnetic radiation emissions from the user, as illustrated in FIG. 8. The subject was both the target and the light source. The visual patterns in the subject's face (left), neck (center), or forearm (right) indicated areas of high emissions versus low emissions. The system can refine the data from this device to extract both movement and physiological data from the emissions output.

In some embodiments, an electromagnetic radiation signal can be attached to a body part of a subject, including, for example, the wrists, ankles, elbows, knees, hips, waist, chest, and head. In some embodiments, electromagnetic radiation sensors can be used to detect electromagnetic radiation. Multiple electromagnetic radiation sensors can be used to measure movement and physiological changes from different positions of view and generate a multi-dimensional data set. Using multiple sensors can provide accurate measurements by reducing the effect of random movement or misalignment of the sensors.

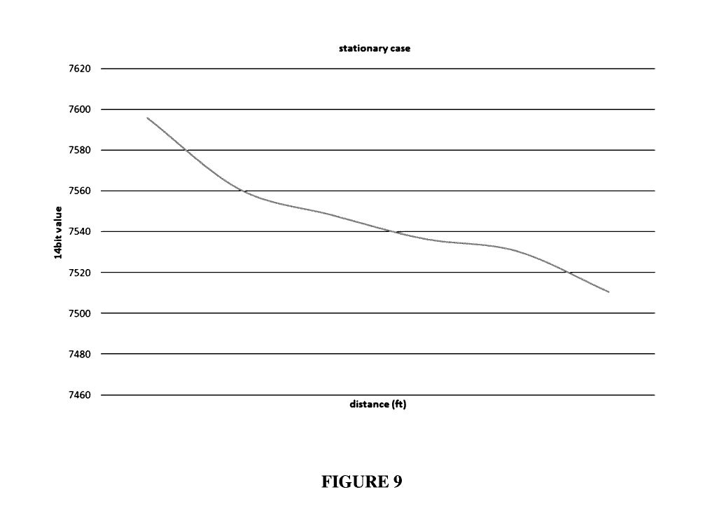

In some embodiments, application-specific algorithms can be used for object tracking. A cascade detection model, which is based on a training type tracking method, can provide good tracking accuracy. The system herein can be used with a robot-mounted thermal target to develop these algorithms iteratively. As shown in FIG. 9, this method uses the measured radiance of the object (measured as photon count) as a function of the object's distance from the detector.

Infrared radiation emissions used and detected in a method of the invention can range from the red edge of the visible spectrum at a wavelength of about 700 nm to about 1 mm, which is equivalent to a frequency of about 430 THz to about 300 GHz. Regions within the infrared spectrum include, for example, near-infrared (NIR), short-wavelength infrared (SWIR), mid-wavelength infrared (MWIR), intermediate infrared (IIR), long-wavelength infrared (LWIR), and far-infrared (FIR). Near-infrared can range from about 0.7 μm to about 1.4 μm, which is equivalent to a frequency of about 214 THz to about 400 THz. Long-wavelength infrared can range from about 8 μm to about 15 μm, which is equivalent to a frequency of about 20 THz to about 37 THz.

In some embodiments, the system can detect infrared radiation with a wavelength of about 700 nm to about 1.5 μm, about 1.5 μm to about 5 μm, about 5 μm to about 10 μm, about 10 μm to about 20 pm, about 20 μm to about 50 pm, about 50 μm to about 100 pm, about 100 μm to about 150 pm, about 150 μm to about 200 pm, about 200 μm to about 250 pm, about 250 μm to about 300 pm, about 300 μm to about 350 pm, about 350 μm to about 400 μm, about 400 μm to about 450 pm, about 450 μm to about 500 pm, about 500 μm to about 550 μm, about 550 μm to about 600 pm, about 600 μm to about 650 pm, about 650 μm to about 700 μm, about 700 μm to about 750 pm, about 750 μm to about 800 pm, about 800 μm to about 850 μm, about 850 μm to about 900 μm, about 900 μm to about 950 μm, or about 950 μm to about 1 mm.

In some embodiments, the system can detect infrared radiation with a wavelength of about 700 nm, about 1.5 μm, about 5 μm, about 10 μm, about 20 μm, about 30 μm, about 40 μm, about 50 μm, about 100 μm, about 150 μm, about 200 μm, about 250 μm, about 300 μm, about 350 μm, about 400 μm, about 450 μm, about 500 μm, about 550 μm, about 600 μm, about 650 μm, about 700 μm, about 750 μm, about 800 μm, about 850 μm, about 900 μm, about 950 μm, or about 1 mm.

In some embodiments, exercise programs, movement, and physiological data can be transmitted to output devices, including, for example, personal computers (PC), such as a portable PC, slate and tablet PC, telephones, smartphones, smart watches, smart glasses, or personal digital assistants.

### EMBODIMENTS

The following non-limiting embodiments provide illustrative examples of the invention, but do not limit the scope of the invention.

### Embodiment 1

A method comprising: a) receiving by a computer system data associated with a first electromagnetic signal from a subject's body, wherein the data associated with the first electromagnetic signal is associated with a gesture of the subject; b) receiving by the computer system data associated with a second electromagnetic signal from the subject's body, wherein the data associated with the second electromagnetic signal is associated with a physiological characteristic of the subject; c) determining by a processor of the computer system based on the data associated with the first electromagnetic signal from the subject's body and the data associated with the second electromagnetic signal from the subject's body a suitable exercise regimen for the subject; and d) outputting the suitable exercise regimen on an output device.

### Embodiment 2

The method of embodiment 1, wherein the first electromagnetic signal is a near-infrared signal.

### Embodiment 3

The method of any one of embodiments 1-2, wherein the second electromagnetic signal is a long-wave infrared signal.

### Embodiment 4

The method of any one of embodiments 1-3, wherein the gesture is a movement of a limb of the subject.

### Embodiment 5

The method of any one of embodiments 1-4, wherein the physiological characteristic is a skin temperature of the subject.

### Embodiment 6

The method of any one of embodiments 1-4, wherein the physiological characteristic is a heart rate of the subject.

### Embodiment 7

The method of any one of embodiments 1-6, further comprising outputting an image of the first electromagnetic signal.

### Embodiment 8

The method of any one of embodiments 1-7, further comprising outputting an image of the second electromagnetic signal.

### Embodiment 9

The method of any one of embodiments 1-8, wherein a source of the first electromagnetic signal is attached to the subject's body.

### Embodiment 10

The method of any one of embodiments 1-9, wherein a source of the second electromagnetic signal is attached to the subject's body.

### Embodiment 11

The method of any one of embodiments 1-8, wherein a source of the first electromagnetic signal is the subject's body.

### Embodiment 12

The method of any one of embodiments 1-8, wherein a source of the second electromagnetic signal is the subject's body.

### Embodiment 13

The method of any one of embodiments 1-12, wherein the first electromagnetic signal is emitted from the subject's body.

### Embodiment 14

The method of any one of embodiments 1-13, wherein the second electromagnetic signal is emitted from the subject's body.

### Embodiment 15

The method of any one of embodiments 1-8, wherein the first electromagnetic signal is emitted by a radiation source to the subject's body, wherein the first electromagnetic signal emitted by the radiation source to the subject's body is reflected off the subject's body prior to detection by a sensor.

### Embodiment 16

The method of any one of embodiments 1-8, wherein the second electromagnetic signal is emitted by a radiation source to the subject's body, wherein the second electromagnetic signal emitted by the radiation source to the subject's body is reflected off the subject's body prior to detection by a sensor.

### Embodiment 17

The method of any one of embodiments 1-16, wherein the subject is a human.

What is claimed is:

1. A method comprising:
a) receiving by a computer system data associated with a first electromagnetic signal from a subject's body, wherein the data associated with the first electromagnetic signal is associated with a gesture of the subject;
b) receiving by the computer system data associated with a second electromagnetic signal from the subject's body, wherein the data associated with the second electromagnetic signal is associated with a physiological characteristic of the subject;
c) determining by a processor of the computer system based on the data associated with the first electromagnetic signal from the subject's body and the data associated with the second electromagnetic signal from the subject's body a suitable exercise regimen for the subject; and
d) outputting the suitable exercise regimen on an output device.

2. The method of claim 1, wherein the first electromagnetic signal is a near-infrared signal.

3. The method of claim 1, wherein the second electromagnetic signal is a long-wave infrared signal.

4. The method of claim 1, wherein the gesture is a movement of a limb of the subject.

5. The method of claim 1, wherein the physiological characteristic is a skin temperature of the subject.

6. The method of claim 1, wherein the physiological characteristic is a heart rate of the subject.

7. The method of claim 1, further comprising outputting an image of the first electromagnetic signal.

8. The method of claim 1, further comprising outputting an image of the second electromagnetic signal.

9. The method of claim 1, wherein a source of the first electromagnetic signal is attached to the subject's body.

10. The method of claim 1, wherein a source of the second electromagnetic signal is attached to the subject's body.

11. The method of claim 1, wherein a source of the first electromagnetic signal is the subject's body.

12. The method of claim 1, wherein a source of the second electromagnetic signal is the subject's body.

13. The method of claim 1, wherein the first electromagnetic signal is emitted from the subject's body.

14. The method of claim 1, wherein the second electromagnetic signal is emitted from the subject's body.

15. The method of claim 1, wherein the first electromagnetic signal is emitted by a radiation source to the subject's body, wherein the first electromagnetic signal emitted by the radiation source to the subject's body is reflected off the subject's body prior to detection by a sensor.

16. The method of claim 1, wherein the second electromagnetic signal is emitted by a radiation source to the subject's body, wherein the second electromagnetic signal emitted by the radiation source to the subject's body is reflected off the subject's body prior to detection by a sensor.

17. The method of claim 1, wherein the subject is a human.
